(19) United States
(12) Patent Application Publication
(10) Pub. No.: US 2012/0123336 A1
(43) Pub. Date: May 17, 2012

(54) PUMP MODULE AND INFUSION PUMP SYSTEM

(76) Inventor: Kenji Kameyama, Kanagawa (JP)

(21) Appl. No.: 13/034,806

(22) Filed: Feb. 25, 2011

(30) Foreign Application Priority Data
Nov. 11, 2010 (JP) 2010-252706

(51) Int. Cl. A61M 5/142

(52) U.S. Cl. 604/151

(57) ABSTRACT

A pump module includes a case; and a pump located in the case for sending a solution according to changes in a volume of a space included in the pump where the solution flows in and out. The space includes first and second openings through which the solution flows in and out, the case includes third and fourth openings through which the solution flows in and out, the third and fourth openings are located at different positions, an angle between a direction in which the solution flows into the third opening and a direction in which the solution flows out from the second opening is greater than or equal to zero degrees and less than 90 degrees, the third opening and the first opening are connected by a first flow path, and the second opening and the fourth opening are connected by a second flow path.

### BACKGROUND OF THE INVENTION

1. Field of the Invention

The present invention relates to a pump module and an infusion pump system using a micro-pump.

2. Description of the Related Art

Conventionally, pumps used in drip infusion apparatuses are relatively large. Thus, even when a portable drip infusion apparatus is used, it is difficult for the patient to freely walk around.

One approach is to use, as the pump, a diffuser type micro-pump including a piezoelectric element. By using such a micro-pump, the patient can move around more easily while being administered intravenous drips, compared to the case of using conventional large-sized pumps.

As described in patent document 1, for example, this type of micro-pump includes a pressure chamber as a solution chamber in which the solution is temporarily stored. An actuator such as a piezoelectric element oscillates any one of the side walls of the pressure chamber, to change the volume of the solution chamber. When the volume of the solution chamber decreases, the solution inside the solution chamber is discharged. When the volume of the solution chamber increases, an additional solution flows into the solution chamber. In this manner, the solution is sent through the drip infusion apparatus.

The solution inside the solution chamber is discharged when the volume of the solution chamber decreases, according to the following principle. As the volume of the solution chamber decreases, the solution in the solution chamber flows to the inlet and the outlet of the pump, toward the outside of the pump. However, according to the configuration of the diffuser provided in the pump, the flow volume in the forward direction (flow volume flowing from the inlet to the outlet) is larger than the flow volume in the backward direction (flow volume flowing from the outlet to the inlet). Therefore, the solution is discharged from the outlet.

However, if the micro-pump described in patent document 1 is actually applied to a drip infusion apparatus, the following problem arises. That is, as the drip infusion apparatus starts operating and the solution flows to the solution chamber, air remains in the solution chamber. Accordingly, the solution chamber cannot be completely filled with the solution.

If air remains in the solution chamber, even if the volume of the solution chamber is changed with the use of the actuator, it may not be possible to send the solution as planned.

For example, it is assumed that the micro-pump as described in patent document 1 is applied as an infusion solution pump used for drip infusion.

The outlet side of the solution chamber in the micro-pump is connected to a catheter such that the flow direction of the infusion solution does not change, and is connected to an injection needle via the catheter. Furthermore, the inlet side of the solution chamber in the micro-pump is connected to another catheter such that the flow direction of the infusion solution does not change, and is connected to an infusion solution bag via the other catheter.

At the time point when the infusion solution pump starts operating, the solution chamber is filled with air, and therefore the micro-pump does not implement its solution sending function. In this case, the micro-pump may be placed at a lower position than the infusion solution bag, so that the infusion solution is sent to the solution chamber by gravity.

However, when the water level in the solution chamber increases while the outlet of the solution chamber is closed, and infusion solution flows into the solution chamber from the upper side, the air moves toward the upper side (inlet side) because the specific gravity of air is lighter than that of the infusion solution. Accordingly, the air and the infusion solution are mixed together, such that the air cannot be completely replaced by the infusion solution. Thus, it is difficult to completely fill the solution chamber with infusion solution without leaving any air (bubbles) in the solution chamber.

One approach is to provide the inlet of the micro-pump on the lower side, and to provide the outlet of the micro-pump on the upper side, so that the infusion solution flows into the solution chamber from the lower side with respect to the direction of gravity. In this method, without closing the outlet of the solution chamber, the infusion solution having heavy specific gravity pushes out the air toward the outlet of the pump as the water level increases. Accordingly, the solution and air are not mixed together, so that bubbles are not generated.

However, in this case, even if the micro-pump is placed at a lower position than the infusion solution bag, the user or nurse needs to hold the micro-pump such that the outlet of the solution chamber surely faces the upper side in the vertical direction. Alternatively, the micro-pump needs to be fixed to an infusion pole such that the outlet of the solution chamber faces the upper side. In either case, the user needs to pay attention to the direction of the solution chamber, i.e., the micro-pump, and therefore the user needs to bear a significant load.
Patent Document 1: Japanese Laid-Open Patent Publication No. 10-110681

### SUMMARY OF THE INVENTION

The present invention provides a pump module and an infusion pump system, in which one or more of the above-described disadvantages are eliminated.

A preferred embodiment of the present invention provides a pump module and an infusion pump system using a micro-pump that sends a solution by changing the volume of a space formed in a substrate made of a material that is easy to process such as silicon, by oscillating the space with an actuator. Specifically, a preferred embodiment of the present invention provides a pump module and an infusion pump system including the pump module, in which a solution chamber has an inlet side that surely faces the lower side in the gravity direction and an outlet side that faces the upper side in the gravity direction when the pump module is normally attached to an infusion pole, so that the solution chamber is filled with the infusion solution without allowing air to remain in the solution chamber when the pump is started to be used, such that the user does not need to bear the load of watching the direction of the solution chamber.

According to an aspect of the present invention, there is provided a pump module including a case; and a pump that sends a solution in accordance with a change in a volume of a space where the solution flows in and out, the space being included in the pump and the pump being located inside the case, wherein the space includes a first opening part through which the solution flows into or out from the space, and a second opening part through which the solution flows out from or into the space, the case includes a third opening part through which the solution flows into or out from the case, and a fourth opening part through which the solution flows out from or into the case, the fourth opening part is located at a different position from the third opening part, an angle between a direction in which the solution flows into or out from the third opening part and a direction in which the solution flows out from or into the second opening part is greater than or equal to zero degrees and less than 90 degrees, the third opening part and the first opening part are connected by a first flow path through which the solution flows into or out from the case and the space, and the second opening part and the fourth opening part are connected by a second flow path through which the solution flows out from or into the space and the case.

According to an aspect of the present invention, there is provided an infusion pump system including an infusion solution container filled with an infusion solution; a needle used for injecting the infusion solution in a biological body; and a pump module that sends the infusion solution toward the needle, the pump module including a case, and a pump that sends the infusion solution in accordance with a change in a volume of a space where the infusion solution flows in and out, the space being included in the pump and the pump being located inside the case, wherein the space includes a first opening part through which the infusion solution flows into or out from the space, and a second opening part through which the infusion solution flows out from or into the space, the case includes a third opening part through which the infusion solution flows into or out from the case, and a fourth opening part through which the infusion solution flows out from or into the case, the fourth opening part is located at a different position from the third opening part, an angle between a direction in which the infusion solution flows into or out from the third opening part and a direction in which the infusion solution flows out from or into the second opening part is greater than or equal to zero degrees and less than 90 degrees, the third opening part and the first opening part are connected by a first flow path through which the infusion solution flows into or out from the case and the space, and the second opening part and the fourth opening part are connected by a second flow path through which the infusion solution flows out from or into the space and the case.

According to one embodiment of the present invention, a pump module and an infusion pump system are provided, in which a solution chamber of a micro-pump provided in the infusion pump system has an inlet side that surely faces the lower side in the gravity direction and an outlet side surely that faces the upper side in the gravity direction when an infusion solution bag to which the infusion pump system is normally attached to an infusion pole, so that the solution chamber is filled with the infusion solution without allowing air to remain in the solution chamber when the micro-pump is started to be used, such that the user does not need to bear the load of watching the direction of the solution chamber.

### BRIEF DESCRIPTION OF THE DRAWINGS

Other objects, features and advantages of the present invention will become more apparent from the following detailed description when read in conjunction with the accompanying drawings, in which:

FIG. 1 illustrates an overview of an infusion apparatus to which a solution sending system according to an embodiment of the present invention is applied;

FIGS. 2A through 2C are schematic diagrams for describing the operation concept of a micro-pump used in an embodiment of the present invention;

FIGS. 3A and 3B are schematic diagrams of an operating state of the micro-pump;

FIGS. 4A and 4B are schematic diagrams of the micro-pump according to an embodiment of the present invention;

FIGS. 5A and 5B illustrate the control unit of the infusion pump system;

FIG. 6 is a flowchart of a first control operation of the infusion pump system according to an embodiment of the present invention;

FIG. 7 is a flowchart of the second control operation of the infusion pump system according to an embodiment of the present invention;

FIG. 8 is a flow chart of a process of performing interruption control when an abnormality occurs;

FIG. 9 is a flow chart of an operation performed by a constricting unit when a system controller is not operating; and

FIG. 10 illustrates a specific example of a flow path resistance changing means for constricting tube.

### DETAILED DESCRIPTION OF THE PREFERRED EMBODIMENTS

A description is given, with reference to the accompanying drawings, of embodiments of the present invention.

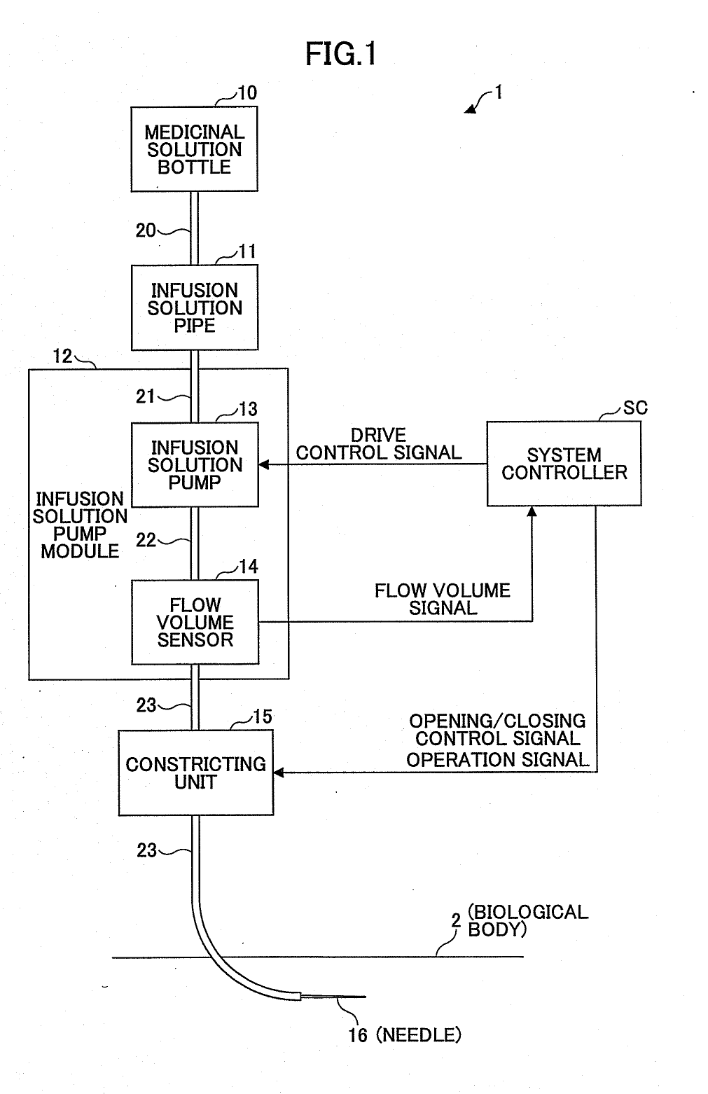

FIG. 1 illustrates an overview of an infusion apparatus to which a solution sending system according to an embodiment of the present invention is applied.

An infusion apparatus 1 includes a medicinal solution bottle (infusion solution container) 10 filled with a medicinal solution or an infusion solution; an infusion solution pipe 11 including one opening connected to the medicinal solution bottle 10 via a tube 20; and a needle 16 that is inserted into a part of a biological body (patient) 2 such as a venous blood vessel for injecting a medicinal solution. Furthermore, the infusion apparatus 1 includes an infusion solution pump module 12 including an infusion solution pump 13 and a flow volume sensor (flow volume detecting unit) 14. The infusion solution pump module 12 is connected to the other opening of the infusion solution pipe 11 via a tube 21, and is connected to the needle 16 via a tube 23. Furthermore, the infusion apparatus 1 includes a constricting unit 15 provided on the tube 23 connecting the infusion solution pump module 12 and the needle 16. The constricting unit 15 is an example of a means for changing the resistance of a flow path (flow path resistance changing means). The constricting unit 15 constricts/compresses the tube 23 from outside to reduce the inner diameter of the tube 23 so that the solution does not flow through the flow path. The constricting unit 15 limits the flow of the medicinal solution by gradually (in a step-by-step manner) increasing the flow path resistance, while allowing a certain amount of fluid to flow through the flow path. The constricting unit 15 facilitates the flow of the medicinal solution inside the tube 23 by gradually (in a step-by-step manner) loosening the constricted state so that the flow path resistance is reduced. Furthermore, the infusion apparatus 1 includes a system controller (control unit) SC that is connected to the infusion solution pump 13, the flow volume sensor 14, and the constricting unit 15, for controlling these respective modules.

In the example of FIG. 1, the infusion solution pump 13 and the flow volume sensor 14 form a single module, i.e., the infusion solution pump module 12; however, the present invention is not so limited. The infusion solution pump 13 and the flow volume sensor 14 may be separate components instead of forming a single module. Furthermore, the tubes in the infusion apparatus 1 are typical catheters used for drip infusion in hospitals, which have elastic, soft properties.

The flow volume sensor 14 is connected to the infusion solution pump 13 via a tube 22. The flow volume sensor 14 measures the flow volume per unit time of the medicinal solution discharged from the infusion solution pump 13, and supplies the measured flow volume as electric signals to the system controller SC.

In the present embodiment, the medicinal solution flows through a flow path extending from the medicinal solution bottle 10 to the needle 16 by passing through the tube 20, the infusion solution pipe 11, the tube 21, the infusion solution pump 13, the tube 22, the flow volume sensor 14, and the tube 23, in the stated order. A constricting part of the constricting unit 15 is provided on the tube 23.

The infusion solution container is not limited to the medicinal solution bottle 10; the infusion solution container may be, for example, a bag type container such as a vinyl bag.

As described in detail below, the infusion solution pump 13 is a diffuser type micro-pump that uses a piezoelectric element. The infusion solution pump 13 receives, from the system controller SC, drive control signals for controlling the driving frequency and the driving voltage (i.e., the driving amplitude) of the piezoelectric element, so that the flow volume of the discharged medicinal solution is controlled.

By using a micro-pump, the pump itself can be made compact, and therefore the patient can move around more easily while being administered intravenous drips, compared to the case of using conventional large-sized pumps.

The flow path resistance changing means may be any kind of means. Examples are a method of directly compressing the tube 23 from the outside of the tube 23 with a movable arm driven by a motor, or a method of compressing the tube 23 with a screw. These elements may be driven with the use of a stepping motor or a regular motor.

Examples of methods of changing the flow path resistance are pressing, twisting, and bending the tube from outside with a gear or a roller.

The flow path resistance changing means may be integrally provided in the infusion solution pump module 12.

A detailed example of the flow path resistance changing means for constricting the tube 23 is described below.

The constricting unit 15 performs control operations as described in detail below, by completely blocking the flow path, or by gradually (in a step-by-step manner) increasing or decreasing the extent of constricting the tube 23 while allowing a certain amount of fluid to flow through the flow path. Accordingly, the resistance in the flow path of the infusion solution is gradually (in a step-by-step manner) increased and decreased.

The constricting unit 15 can be removed from the tube 23, or the constricting unit 15 can be integrally provided in the infusion solution pump module 12. Therefore, the constricting unit 15 may be always provided for a patient who requires such a means (a patient that is expected to move around during the drip infusion). Meanwhile, the constricting unit 15 may not be provided for a patient who does not require such a means (a patient that is not expected to move around during the drip infusion). Accordingly, operating costs can be reduced.

Furthermore, the constricting unit 15 constricts the tube 23 by sandwiching the tube 23 from outside, and therefore the infusion solution does not contact the constricting unit 15. Accordingly, the constricting unit 15 can be repeatedly reused.

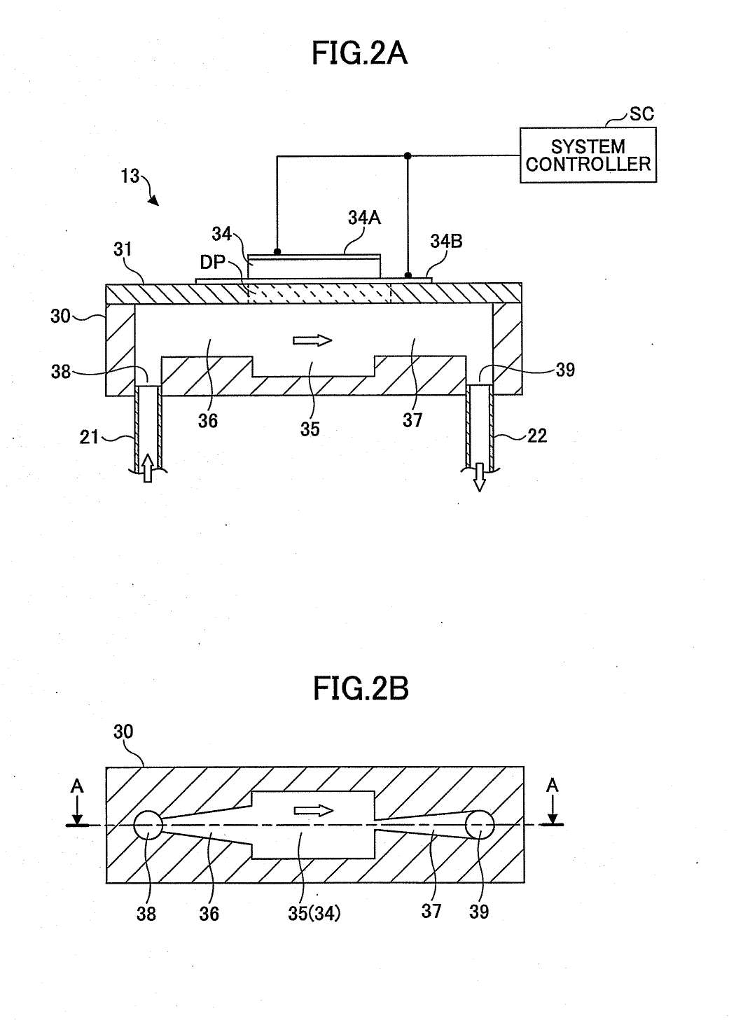

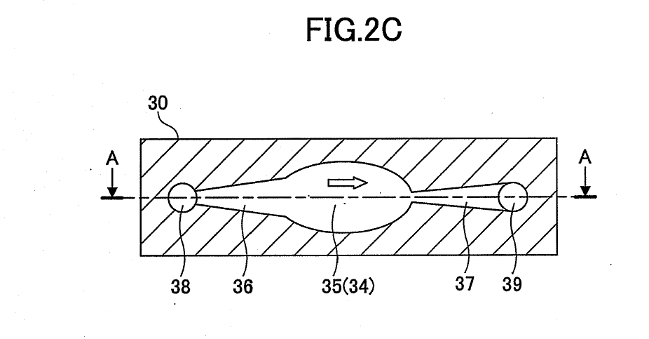

FIGS. 2A through 2C are schematic diagrams for describing the operation concept of the infusion solution pump 13 used in an embodiment of the present invention. FIG. 2A is a cross-sectional view of the infusion solution pump 13 and FIGS. 2B and 2C are plan views of the infusion solution pump 13. FIG. 2A is a cross-sectional view of the infusion solution pump 13 cut along a line A-A in FIGS. 2B and 2C.

The cross-sectional shape of a solution chamber 35 is not limited to a rectangular shape as illustrated in FIG. 2B; the cross-sectional shape of the solution chamber 35 may be a round shape as illustrated in FIG. 2C.

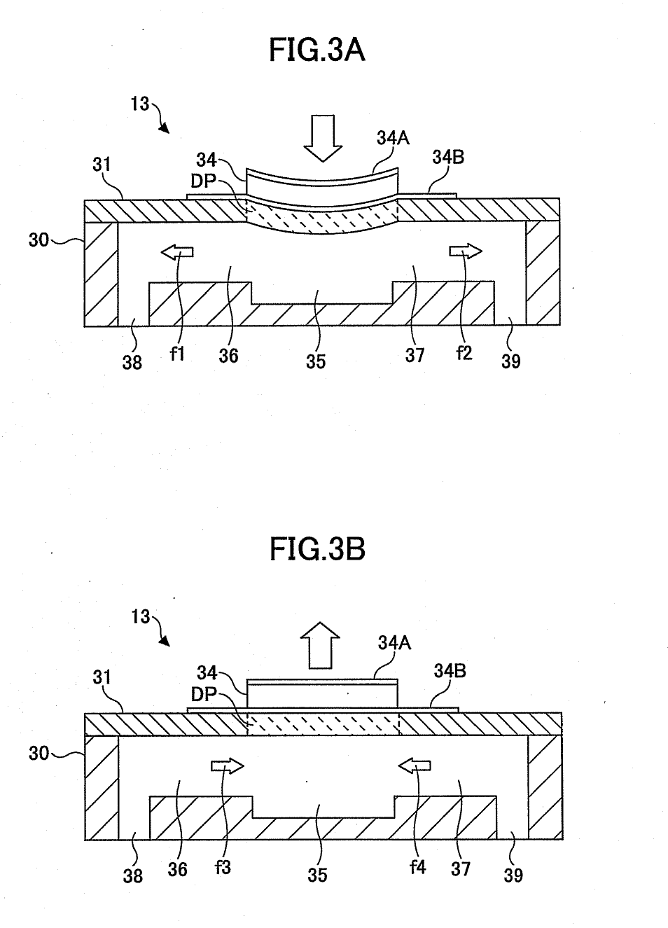

Furthermore, FIGS. 3A and 3B are schematic diagrams of an operating state of the infusion solution pump 13.

The infusion solution pump 13 primarily includes a Si (silicon) substrate 30 in which a groove is formed by etching, and a glass substrate (plate member) 31 that is anodically-bonded to the silicon substrate 30.

A space formed by the groove provided in the silicon substrate 30 and the glass substrate 31 acts as a pressure chamber (pump chamber) 35. A piezoelectric element 34 is provided on the top surface of the glass substrate 31, at a position corresponding to the solution chamber 35. Diffusers 36 and 37 are formed by etching in the silicon substrate 30 along a direction in which the fluid progresses in the solution chamber 35. The diffusers 36 and 37 are flow paths having a cross-sectional area that gradually increases.

The piezoelectric element 34 includes electrodes 34A and 34B on opposite sides of the piezoelectric element 34 (the electrodes 34A and 34B are provided on the sides of the piezoelectric element 34 that are configured to bend). Furthermore, the piezoelectric element 34 is provided on the glass substrate 31 via the electrode 34B.

Furthermore, an inlet 38 and an outlet 39 are through holes that are respectively connected to the diffuser 36 and the diffuser 37, in such a manner that fluid can flow through. The inlet 38 and the outlet 39, which respectively act as the inlet and the outlet of the solution chamber 35, are formed by etching in the silicon substrate 30. The tube 21 is connected to the inlet 38 in such a manner that fluid can flow in from the infusion solution pipe 11. The tube 22 is connected to the outlet 39 in such a manner that fluid can flow out to the flow volume sensor 14. The solution chamber 35 is connected to the tube 21 and the tube 22 in such a manner that fluid can flow through, so that the solution chamber 35 acts as a part of the flow path of the constricting unit 15.

As a driving voltage (voltage pulse) is applied to the piezoelectric element 34 from the system controller SC, the piezoelectric element 34 bends. Accordingly, the part of the glass substrate 31 that contacts the piezoelectric element 34 operates as a diaphragm part DP, so that pressure is applied to the solution chamber 35. Thus, the solution chamber 35 contracts (see FIG. 3A) and expands (see FIG. 3B). As the solution chamber 35 contracts and expands, the pressure levels in the diffuser 36 and the diffuser 37 become different. Consequently, the fluid is caused to flow.

To apply the driving voltage to the piezoelectric element 34, the system controller SC applies a voltage between the electrodes 34A and 34B. A positive voltage is applied to the electrode 34A, and the electrode 34B is connected to GND. The difference in potential between the electrodes 34A and 34B acts as the driving voltage for driving the piezoelectric element 34.

As the solution chamber 35 repeats contracting and expanding, a steady flow of fluid flowing from the inlet 38 to the outlet 39 is generated.

More specifically, as shown in FIG. 2B, the cross-sectional area of the diffuser 36 gradually increases from the inlet 38 to the solution chamber 35. Furthermore, the cross-sectional area of the diffuser 37 gradually increases from the solution chamber 35 to the outlet 39. That is to say, the cross-sectional areas of the diffuser 36 and the diffuser 37 gradually increase in a direction indicated by an arrow in FIG. 2B.

By applying a voltage pulse to the piezoelectric element 34, the diaphragm part DP can be oscillated. That is to say, by applying a voltage pulse to the piezoelectric element 34, the solution chamber 35 repeatedly contracts and expands (expanding meaning expanding from the contracted state). The contraction ratio of the solution chamber 35 (the extent to which the diaphragm part DP bends) is determined by the pulse amplitude and pulse width of the voltage applied to the piezoelectric element 34. The number of times the solution chamber 35 repeatedly contracts/expands is determined by the frequency of the voltage pulse.

When the solution chamber 35 expands (actually, the expansion ratio is 1), the medicinal solution flows into the solution chamber 35 from both the inlet 38 and the outlet 39.

The fluid that flows into the solution chamber 35 from the inlet 38 and the outlet 39 passes through the diffuser 36 and the diffuser 37, respectively. As described above, the cross-sectional area of the diffuser 36 and the diffuser 37 gradually increases in the direction indicated by the arrow in FIG. 2B. Therefore, in the diffuser 36 and the diffuser 37, a small resistance is applied to the fluid flowing in the direction indicated by the arrow in FIG. 2B, while a large resistance is applied to the fluid flowing in a direction opposite to the direction indicated by the arrow in FIG. 2B.

Accordingly, in the state illustrated in FIG. 3A, a medicinal solution f1 that is discharged toward the inlet 38 flows in a direction in which the cross-sectional area of the diffuser 36 decreases. Therefore, the resistance is high and the flow volume is low. Meanwhile, a medicinal solution f2 that is discharged toward the outlet 39 flows in a direction in which the cross-sectional area of the diffuser 37 increases. Therefore, the resistance is low and the flow volume is large.

Furthermore, in the state illustrated in FIG. 3B, a medicinal solution f3 that flows in from the inlet 38 flows in a direction in which the cross-sectional area of the diffuser 36 increases. Therefore, the resistance is low and the flow volume is large. Meanwhile, a medicinal solution f4 that flows in from the outlet 39 flows in a direction in which the cross-sectional area of the diffuser 37 decreases. Therefore, the resistance is high and the flow volume is small.

When the solution chamber 35 contracts and expands once, the net amount of fluid flowing from the inlet 38 to the solution chamber 35 is |f3−f1|, while the net amount of fluid flowing from the solution chamber 35 to the outlet 39 is |f2−f4|. Therefore, the net amount of fluid flowing from the inlet 38 to the outlet 39 is f=|f1−f3|=|f4−f2|.

Assuming that the solution chamber 35 has a volume W and a contraction ratio β, the equation f=W(1−β) is satisfied. As the solution chamber 35 repeats contracting and expanding, a steady flow of fluid flowing from the inlet 38 to the outlet 39 is generated. Assuming that the number of times (frequency) that the solution chamber 35 repeats contracting and expanding is ω, a fluid having a volumetric flow volume of F=ωf=ωW(1−β) per unit time flows from the inlet 38 to the outlet 39.

The volumetric flow volume F can be controlled by adjusting at least one of a pulse amplitude V, a pulse width H (pulse area VH), and a pulse period T (frequency 1/T) of the voltage pulse applied to the piezoelectric element 34.

By increasing (or decreasing) the pulse width V (or pulse area VH) of the voltage pulse applied to the piezoelectric element 34, the extent to which the piezoelectric element 34 contracts and expands, i.e., the extent to which the diaphragm part DP bends, increases (or decreases). Therefore, by changing the pulse width V (or pulse area VH), the expansion/contraction ratio (1−β) of the solution chamber 35 can be adjusted. Accordingly, the flow volume F=ωW(1−β) can be controlled. Furthermore, by increasing (or decreasing) the frequency of the voltage pulse, the frequency of oscillation of the diaphragm part DP (i.e., the frequency ω that the solution chamber 35 repeats contracting/expanding per unit time) increases (or decreases). Accordingly, by changing the frequency of the voltage pulse, the frequency ω that the solution chamber 35 repeats contracting/expanding per unit time can be adjusted.

However, the structure of the micro-pump is not so limited. For example, it is possible to use a pump capable of sending fluid by the following structure. Specifically, even if the diffusers 36 and 37 are not provided, it is possible to provide a valve in one or both of the inlet 38 and the outlet 39. The valve opens only in the desired direction of the fluid flow. Furthermore, the volume of the solution chamber 35 is variable.

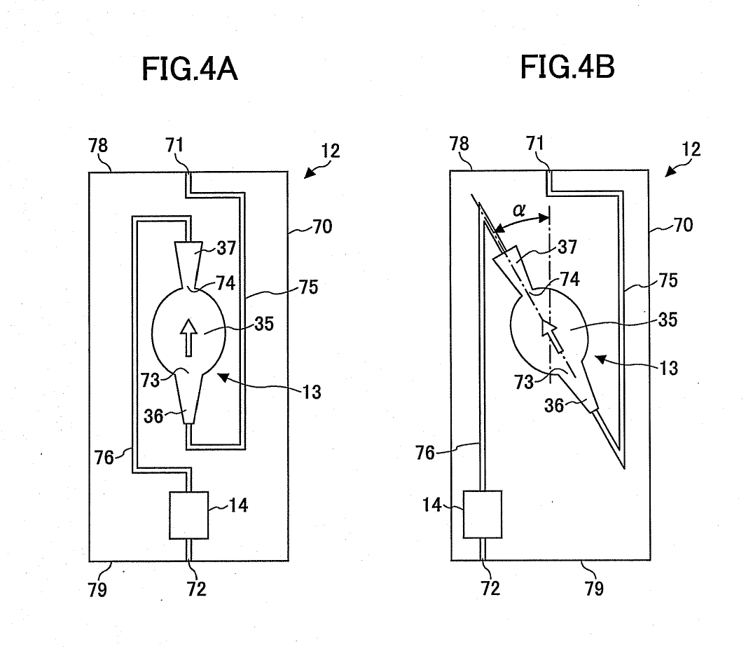

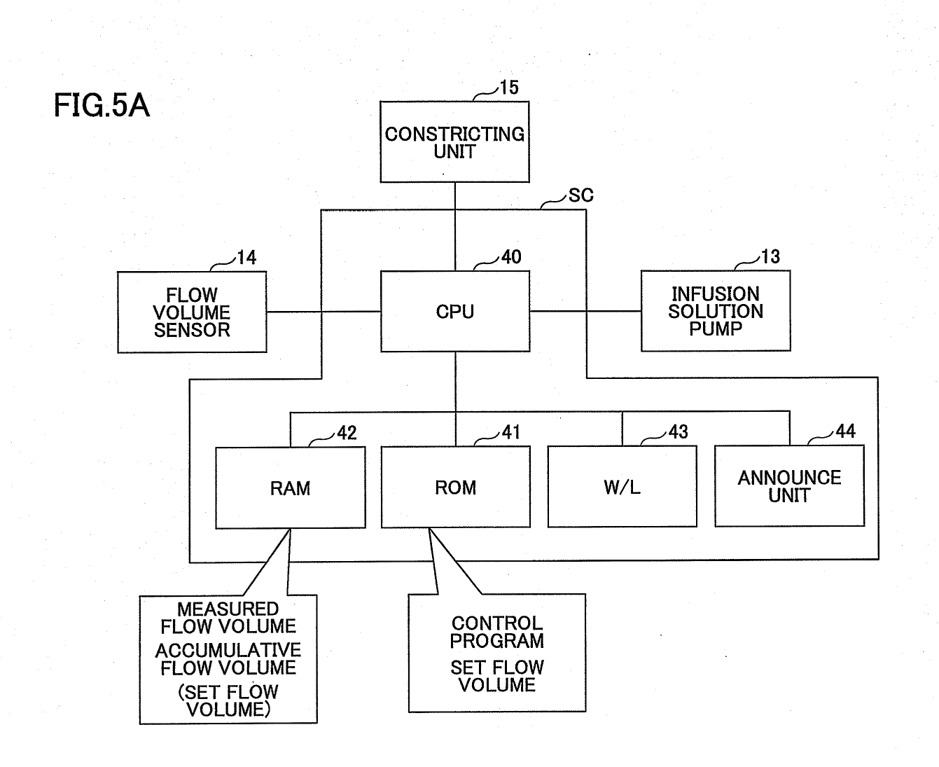

FIGS. 4A and 4B are schematic diagrams of the infusion solution pump module 12 according town embodiment of the present invention.

The infusion solution pump module 12 according to an embodiment of the present invention includes the infusion solution pump 13 that is covered by a case 70 made of plastic. The infusion solution pump 13 is made by forming the solution chamber 35 and the diffusers 36 and 37 in a silicon substrate. The solution chamber 35 is a space where the solution (primarily a medicinal solution and an infusion solution) enters and exits. Furthermore, the infusion solution pump 13 includes a piezoelectric element provided on the glass substrate of the solution chamber 35, and a driving circuit for driving the piezoelectric element according to signals received from the system controller SC.

Furthermore, on the outer wall of the case 70, there is provided an opening part (third opening part) 71 and an opening part (fourth opening part) 72. The opening part 71 is provided on the inlet side to which the medicinal solution or infusion solution flows in from the medicinal solution bottle 10 including the medicinal solution or infusion solution, via the tube 21. The opening part 72 is provided on the outlet side from which the medicinal solution or infusion solution flows out to the needle 16, via the tube 23. In the present embodiment, the case 70 is a rectangular parallelepiped having six faces. The opening part 71 is provided on the opposite side to the side on which the opening part 72 is provided. However, the case 70 is not limited to having six faces; the case 70 may have eight or nine faces, or may have a spherical shape overall with one face.

In the examples of FIGS. 4A and 4B where the case 70 is a rectangular parallelepiped, the opening part 71 on the inlet side is provided on one wall 78 in the longitudinal direction of the case 70, and the opening part 72 on the outlet side is provided on a wall 79 on the opposite side of the wall 78.

However, the opening part 72 does not necessarily need to be provided on the face opposite to the face with the opening part 71. However, by providing the opening part 72 on the face opposite to the face with the opening part 71, the solution outwardly appears to be linearly flowing from the opening part 71 to the opening part 72, regardless of the flow direction inside the case 70.

Furthermore, the solution chamber 35 has an opening part (first opening part) 73 and an opening part (second opening part) 74. The opening part 73 is where the solution flows into the solution chamber 35 via the diffuser 36 on the inlet side. The opening part 74 is where the solution flows out from the solution chamber 35 toward the diffuser 37 on the outlet side. The inner diameter of the diffuser 36 is larger on the opening part 73 side of the solution chamber 35, than on the side of a flow path 75. Furthermore, the inner diameter of the diffuser 37 is smaller on the opening part 74 side of the solution chamber 35, than on the side of a flow path 76. The solution sending function of the micro-pump is implemented in a direction extending from the opening part 73 to the opening part 74.

When the medicinal solution bottle 10 to which the infusion solution pump module 12 is connected via the tube 21 is hung on an infusion pole, the medicinal solution bottle 10 is held at a position higher than that of the infusion solution pump module 12. That is to say, the case 70 is held in such a manner that the opening part 71 is facing upward in the vertical direction. In this state, the opening part 74 through which the solution flows out from the solution chamber 35 is also facing upward in the vertical direction. The infusion solution pump 13 is disposed in the case 70 such that the diffuser 36 is lower than the diffuser 37 in the vertical direction. More specifically, the infusion solution pump 13 is disposed in the case 70 such that the angle between the direction in which the solution flows into the opening part 71 and the direction in which the solution is discharged from the opening part 74 is greater than or equal to zero degrees and less than 90 degrees. FIG. 4A illustrates a case where the direction in which the solution flows to the opening part 71 and the direction in which the solution is discharged from the opening part 74 is zero degrees. The direction in which the solution flows to the opening part 71 and the direction in which the solution is discharged from the opening part 74 does not need to be zero; as long as such an angle is greater than or equal to zero degrees and less than 90 degrees, the same effects can be achieved as bubbles in the solution chamber 35 can exit from the solution chamber 35 by buoyancy. For example, FIG. 4B illustrates a case where the direction in which the solution flows to the opening part 71 and the direction in which the solution is discharged from the opening part 74 is 45 degrees. In this case, the bubbles in the solution chamber 35 rise in the solution chamber 35 by buoyancy, and exit from the solution chamber 35 through the opening part 74. Considering the effects from a different point of view, if the angle between the direction in which the solution flows into the opening part 71 and the direction in which the solution is discharged from the opening part 74 is greater than or equal to zero degrees and less than 90 degrees, when the solution flows into the solution chamber 35 through the opening part 73 when the solution chamber 35 is filled with air, the water level rises toward the opening part 74. Therefore, it is possible to prevent bubbles from being generated in the solution chamber 35. Furthermore, in the infusion solution pump module 12 illustrated in FIGS. 4A and 4B, the opening part 74 of the solution chamber 35 to which the solution is sent, is disposed closer to the opening part 71 at the inlet of the case 70, than is the opening part 73 through which the solution enters the solution chamber 35. Therefore, in the present embodiment, the opening part 71 is formed on the wall facing the opening part 74 to which the solution is sent. In other words, the micro-pump is disposed in the case 70 in such a manner that the diffuser 37 is closer to the opening part 71, than is the diffuser 36. In the present embodiment, the opening part 74 is facing toward the opening part 71.

In the case 70, the opening part 71, the opening part 74, the opening part 73, and the opening part 72 are provided in the stated order from the top in the vertical direction. These openings are formed along a substantially straight line.

Thus, in the case 70 having the opening part 71 on the inlet side that is facing upward, the infusion solution pump 13 (solution chamber 35) is sending the infusion solution from the lower side to the upper side in the vertical direction (gravity direction). The opening part 71, through which the infusion solution flows into the infusion solution pump module 12, is provided on a wall positioned on the downstream side in the infusion solution sending direction with respect to the opening part 74 that is on the outlet side of the solution chamber 35, i.e., the opening part 71 is provided on the outer wall of the case 70.

Therefore, assuming that the solution discharging (solution sending) direction extends from the top to the bottom in the vertical direction of the infusion solution pump module 12, the solution sending direction of the infusion solution pump 13 (solution chamber 35) in the case 70 appears to be in a direction opposite to the solution sending direction of the infusion solution pump module 12.

Particularly, in the example shown in FIG. 4A, the opening part (third opening part) 71 on the inlet side of the case 70 is provided on a wall positioned on the downstream side along an extended line of the solution discharging direction with respect to the opening part (second opening part) 74 that is on the outlet side of the solution chamber 35.

That is to say, the opening part 74 that is on the outlet side of the solution chamber 35 is facing toward the opening part 71 on the inlet side of the case 70. The opening part 73 on the inlet side of the solution chamber 35 is facing the opening part 72 on the outlet side of the case 70.

Specifically, the inlet of the solution chamber 35 is provided on the outlet side of the case 70, and the outlet of the solution chamber 35 is provided on the inlet side of the case 70. That is to say, the opening part 73 on the inlet side of the infusion solution pump 13 is near the wall 79 on the outlet side of the case 70, and the opening part 74 on the outlet side of the infusion solution pump 13 is near the wall 78 on the inlet side of the case 70.

In both FIGS. 4A and 4B, the openings of the solution chamber 35 and the openings of the case 70 have the above positional relationships. Additionally, in the case 70, the solution from the opening part 71 of the case 70 is sent to the opening part 73 (diffuser 36) of the solution chamber 35 via the flow path 75. Furthermore, the opening part 72 receives the solution that is sent from the opening part 74 (diffuser 37) of the solution chamber 35 via the flow path 76 and the flow volume sensor 14, in place of the tube 22 in FIG. 1. That is to say, the flow path 75 is for connecting the opening part 71 and the diffuser 36. Furthermore, the flow path 76 is for connecting the opening part 72 and the diffuser 37, in place of the tube 22.

As described above, the infusion solution pump 13 is disposed in the case 70 in an opposite direction (upside down) with respect to the solution sending direction, i.e., in an opposite direction to the solution sending direction of the tubes 21 and 22 connected to the infusion solution pump 13. Therefore, in the case 70, the flow path 75, which connects the opening part 71 and the opening part 73 of the solution chamber 35, curves around to be connected to the opening part 73 from below. Furthermore, the flow path 76, which connects the opening part 74 and the opening part 72, extends from the opening part 74 and curves around in the solution chamber 35 to be connected to the opening part 72.

In the present embodiment, the flow paths 75 and 76 are made of tube-like members; however, the present invention is not so limited. A known configuration for guiding a fluid in a desired direction may be used. Furthermore, instead of providing the diffusers 36 and 37, there may be a valve provided in one or both of the inlet 38 and the outlet 39. The valve opens only in the direction in which the solution is to be provided. With the use of such a valve, the volume in the solution chamber 35 may be variable, so that the pump has a solution sending function.

When the above described infusion solution pump module 12 is applied to the drip infusion apparatus as illustrated in FIG. 1, the medicinal solution bottle 10 is hung to an infusion pole, so that the medicinal solution bottle 10 is held at a higher position than that of the needle 16. Furthermore, the opening part 71 of the case 70 is connected to the medicinal solution bottle 10 via the tube 21, and the infusion solution pump module 12 is hung in a substantially vertical direction. Accordingly, the infusion solution from the medicinal solution bottle 10 enters the infusion solution pump 13 through the opening part 71, passes through the flow path 75, and enters the solution chamber 35 from below.

Therefore, as described in the background of the invention, by supplying the infusion solution from below the solution chamber 35, the air in the solution chamber 35 can be pushed out of the opening part 74 that is facing upward, and the solution chamber 35 can be filled with the infusion solution such that bubbles are not generated.

Furthermore, even if a slight amount of bubbles are generated, the bubbles can be pushed out from the opening part 74, so that the infusion solution pump 13 is prevented from stopping. The bubbles that have been pushed out can be easily trapped by providing an air trap outside the infusion solution pump 13.

With the infusion solution pump module 12 according to an embodiment of the present invention, the nurse etc. does not need to consider the direction of the opening part on the outlet side of the solution chamber 35. The nurse simply needs to connect, to the medicinal solution bottle 10, the tube 21 that is connected to the opening part 71 of the infusion solution pump module 12, and hang the infusion solution pump module 12 in a substantially vertical direction. Therefore, the load on the nurse is significantly reduced.

The infusion solution pump 13 according to an embodiment of the present invention has a configuration as illustrated in FIGS. 4A and 4B. The infusion solution pump module 12 may be connected in an opposite direction to the above, i.e., by connecting the opening part 72 to the medicinal solution bottle 10 and by connecting the opening part 71 to the needle 16. Accordingly, the opening part 72 is connected, via the flow path 76, to the diffuser 37 (opening part 74) of the infusion solution pump 13 to which the solution is sent. Thus, if the medicinal solution bottle 10 is held at a higher position than the infusion solution pump 13, according to the function of the infusion solution pump 13, it is possible to control the flow of the infusion solution flowing in through the opening part 72, while sending the solution from the opening part 72 to the opening part 71.

The opening part 71 and the opening part 72 of the case 70 are formed in walls that are furthest away from each other, i.e., on opposite walls.

Accordingly, the tubes connected to the opening part 71 and the opening part 72 extends linearly, which is advantageous in terms of appearance.

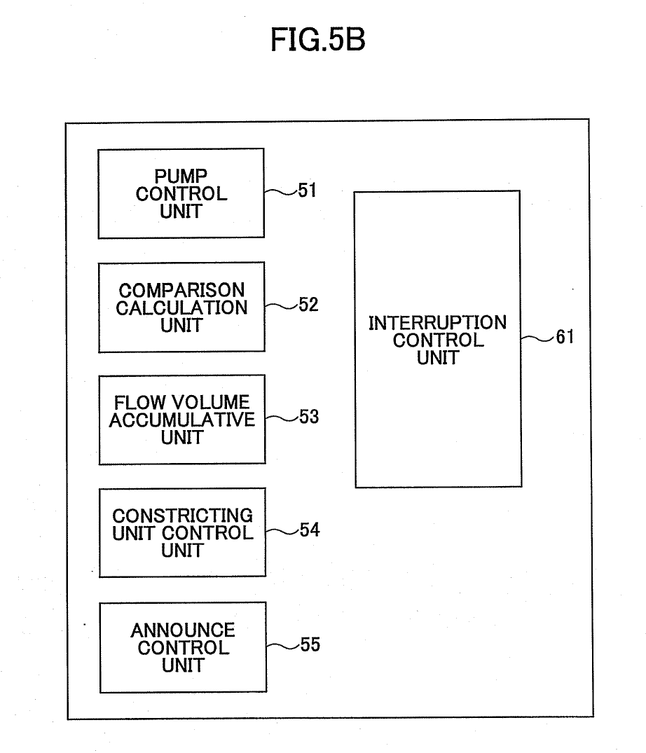

FIGS. 5A and 5B illustrate the control unit of the infusion pump system (infusion apparatus 1). FIG. 5A is a hardware block diagram and FIG. 5B illustrates a control program executed by the control unit.

As shown in FIG. 5A, the system controller SC includes a CPU 40; a ROM (Read Only Memory) 41 for storing a control program and data relevant to an ideal flow volume of the medicinal solution per unit time as the a predetermined set value (hereinafter, set flow volume); and a RAM (Random Access Memory) 42 for loading the control program read from the ROM 41 and for being used as a work area for temporarily storing flow volume data that is a detected value acquired from the flow volume sensor 14 (hereinafter, measured flow volume) and calculated data.

Furthermore, the system controller SC includes a wireless (W/L) communications unit 43 for transmitting a signal to a nurse when there is an abnormality in the infusion pump system (infusion apparatus 1); and an announce unit 44 that announces such an abnormality by emitting light from an LED.

Instead of storing the set flow volume in the ROM 41, the set flow volume may be stored in the RAM 42 by using an input unit to appropriately input a value in accordance with the medicine and the state of the patient.

As described above, the system controller SC is electrically connected to the flow volume sensor 14, the constricting unit 15, and the infusion solution pump 13.

The CPU 40 receives measured flow volume data from the flow volume sensor 14 and compares the measured flow volume with the set flow volume. When the measured flow volume is higher than the set flow volume, the CPU 40 changes the pulse amplitude, the pulse width, and the pulse period of the voltage pulse applied to the piezoelectric element 34 of the infusion solution pump 13 described with reference to FIGS. 2A through 3B, to adjust the flow volume.

Furthermore, as shown in FIG. 5B, the CPU 40 executes a pump control unit 51 that controls the infusion solution pump 13 to change the flow volume of the discharged fluid or to stop the operation of the infusion solution pump 13; a comparison calculation unit 52 that compares the set flow volume with the measured flow volume of the fluid; a flow volume accumulative unit 53 that accumulates the measured flow volume and calculates the total amount of medicinal solution that has been infused; a constricting unit control unit 54 that controls the constricting unit 15 to open or block the tube 23; an announce control unit 55 that makes an announcement to a nurse or an external device by controlling the announce unit 44 and the wireless communications unit 43, when the constricting unit 15 has constricted the tube 23 or when the constricting unit 15 cannot normally (properly) constrict the tube 23 in a diagnosis operation described below; and an interruption control unit 61 interrupts processes performed by the respective units and stops the operation of the infusion solution pump 13 and operates the constricting unit 15 when an abnormality occurs in any part of the infusion pump system (infusion apparatus 1).

Next, a description is given of an operation of controlling the flow volume in the infusion pump system (infusion apparatus 1) according to an embodiment of the present invention.

After being started up, the system controller SC reads the total amount of infusion solution and an infusion solution rate (flow volume) per unit time that has been set in advance. Next, the system controller SC starts driving the infusion solution pump 13 in accordance with an instruction to start drip infusion that is input with the use of an operation unit (not shown) provided in the system controller SC.

The basic operations are as follows. The system controller SC reads, as the measured flow volume, signals output from the flow volume sensor 14. The comparison calculation unit 52 compares the measured flow volume with the flow volume set in advance (set flow volume). The pump control unit 51 adjusts at least one of the pulse amplitude, the pulse width, and the pulse period of the voltage pulse applied to the piezoelectric element 34, in order to control the operations of the infusion solution pump 13 so that the measured flow volume and the set low volume become the same.

At the same time, the constricting unit control unit 54 accumulates the flow volume per unit time to calculate the amount of infusion solution injected in the biological body.

The pump control unit 51 compares a predetermined total amount of infusion solution to be injected with the accumulative flow volume value. When the accumulative flow volume value has not reached the predetermined total amount, the pump control unit 51 continues operating the infusion solution pump 13. However, when the accumulative flow volume value has reached the predetermined total amount, the pump control unit 51 stops the operation of the infusion solution pump 13, and ends the drip infusion operation.

However, when the system controller SC cannot obtain any signals from the flow volume sensor 14, or when the measured flow volume indicates an abnormally high value that is usually inconceivable, it is highly likely that an external failure (e.g., the needle falls out, extravascular administration is performed, a shock is applied, the temperature changes rapidly, and the position of the medicinal solution bottle changes rapidly) has occurred in an element of the infusion pump system (infusion apparatus 1) (e.g., the tubes 11, 21, 22, 23, the infusion solution pipe 11, the infusion solution pump 13, the flow volume sensor 14, and the medicinal solution bottle 10). In this case, the flow volume cannot be changed to the set flow volume by controlling the infusion solution pump module 12.

In such a case, the interruption control unit 61 interrupts the control operations. Specifically, regardless of the program being executed, the constricting unit 15 constricts the tube 23 and the interruption control unit 61 forcibly stops the pumping operation. Accordingly, when the above-mentioned abnormalities occur, the flow path can be immediately blocked, so that any serious accidents can be prevented before they occur.

Even if the measured flow volume is not an abnormal value, if the solution sending flow volume becomes greater than or equal to a set value, a regular closed-loop control operation is performed on the infusion solution pump 13, so that the infusion solution pump 13 is driven under conditions for decreasing the flow volume. When the detection value acquired by the flow volume sensor 14 decreases, and once again reaches the set flow volume (or becomes included within a predetermined margin of error with respect to the set flow volume), the regular closed-loop control operation is completed.

Meanwhile, when the solution sending flow volume cannot be controlled to reach the set value even if the driving conditions of the infusion solution pump 13 are changed, the following factor may be assumed. That is, the height of the position of the medicinal solution bottle 10 may have largely changed from the originally intended position. Accordingly, the infusion solution may be flowing due to gravity, such that the flow volume is outside the range that is controllable by the infusion solution pump 13.

In this case, in the present embodiment, the pump control unit 51 stops driving the infusion solution pump 13 and waits for a predetermined length of time (to remove any impact on the flow volume that flows according to inertia from driving the infusion solution pump 13). Then, the pump control unit 51 detects the measured flow volume output by the flow volume sensor 14, as the flow volume of the infusion solution caused by gravity applied on the infusion solution.

The constricting unit control unit 54 controls the constricting unit 15 to constrict the tube 23 so that the flow volume of infusion solution according to gravity is reduced, and the flow path is constricted by at least an extent such that the measured flow volume can be controlled to reach the set volume when the infusion solution pump 13 is driven.

Accordingly, it is possible to minimize the impact of gravity on the flow volume of the infusion solution, so that the flow volume can be reduced to a level that can be controlled by the infusion solution pump 13.

In this case, the relationship between the extent of constricting the tube 23 and the flow volume is stored as a table in the ROM 41, and the values can be compared to accurately adjust the flow volume of the infusion solution caused by gravity.

After detecting the flow volume of the infusion solution caused by gravity, operation of the infusion solution pump 13 is resumed, and the operations of constricting the tube 23 and controlling the infusion solution pump 13 are simultaneously performed. Accordingly, the flow volume can be controlled to be a normal flow volume within a short period of time.

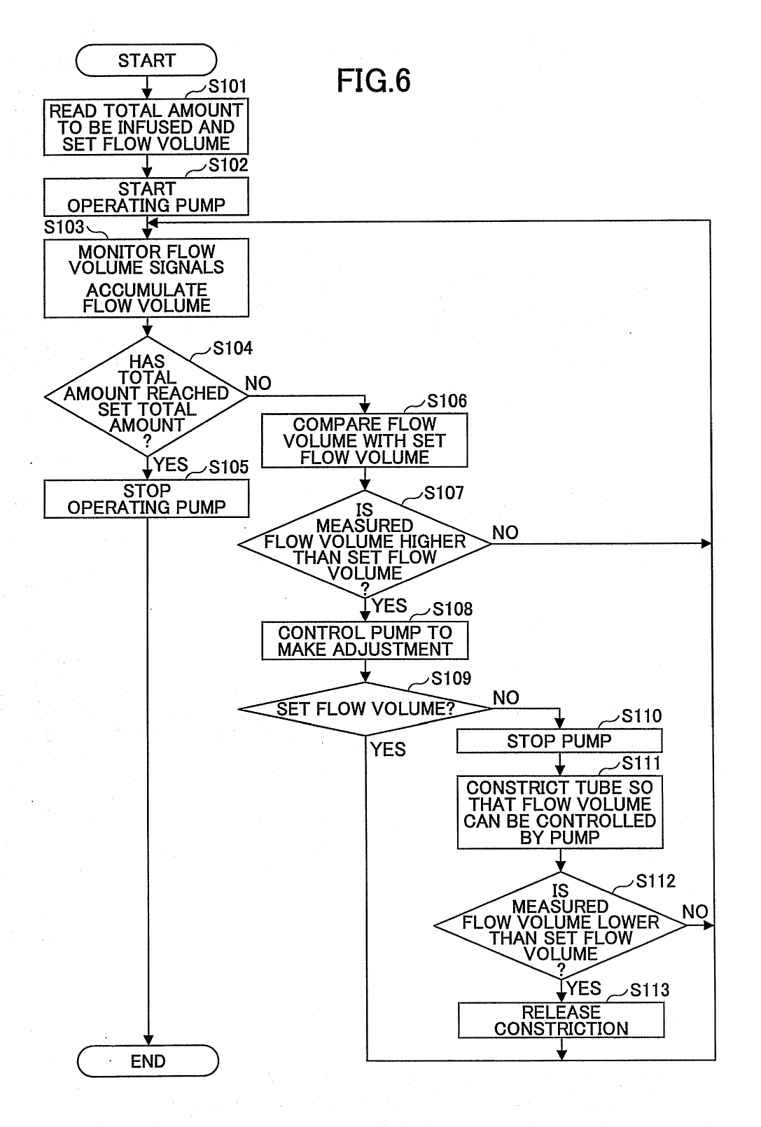

FIG. 6 is a flowchart of a first control operation of the infusion pump system (infusion apparatus 1) according to an embodiment of the present invention.

For every predetermined time period, the system controller SC compares the sensor flow volume (measured flow volume) with a predetermined threshold, and detects an abnormality when the sensor flow volume exceeds the threshold.

When the state of the flow volume sensor 14 is normal, and the flow volume is zero, the flow volume sensor 14 outputs signals of 2.5 V to the system controller SC. However, when the output signal is lower than 2.5 V, or when the output signal is 0 V, it is determined that a problem has occurred in the flow volume sensor 14.

The following is a description of a process flow when there are no problems in the output signals or the measured flow volume of the flow volume sensor 14.

When the infusion pump system 1 starts operating, the CPU 40 reads a predetermined total amount of infusion solution (to be infused) and the ideal flow volume per unit time from the ROM 41 (step S101).

Next, the CPU 40 issues a command to operate the infusion solution pump 13 (step S102).

The CPU 40 constantly monitors the flow volume obtained based on signals input from the flow volume sensor 14. Furthermore, the CPU 40 monitors the value of the flow volume sensor 14, and accumulates the total amount of medicinal solution that has flown through the infusion solution pump 13 based on the value of the flow volume sensor 14 (step S103). When the CPU 40 determines that the total amount has reached the predetermined total amount read in step S101 (YES in step S104), it means that the drip infusion has been completed, and therefore the CPU 40 stops the operation of the infusion solution pump 13 (step S105).

When the CPU 40 determines that the total amount has not reached the total amount read in step S101 (NO in step S104), for every predetermined time period, the CPU 40 compares the flow volume obtained based on the value of the flow volume sensor 14 with the set flow volume acquired in step S101 (step S106).

When the measured flow volume is higher than the set flow volume (YES in step S107), the CPU 40 controls the infusion solution pump 13 to increase/decrease/adjust the flow volume by changing the frequency and the driving voltage of the infusion solution pump 13 (step S108).

When the measured flow volume becomes within a threshold range with respect to the set flow volume by performing the control operation (YES in step S109), it is determined that the variation is within a closed-loop control operation, and the process returns to step S103.

However, when the variation amount exceeds a certain value although it is not an abnormal value, the flow volume cannot be adjusted simply by controlling the infusion solution pump 13. A variation of this extent is considered to be caused not only by a problem in the infusion solution pump 13, but also by the impact of gravity, which arises when the height of the position of the medicinal solution bottle 10 changes more than expected.

In an embodiment of the present invention, when the measured flow volume does not become the set volume by controlling the infusion solution pump 13 (NO in step S109), the flow of the infusion solution caused by gravity is adjusted as follows.

The CPU 40 temporarily stops the infusion solution pump 13 (step S110).

At this point, the flow volume sensor 14 is still operating. Therefore, the CPU 40 can obtain, from signals from the flow volume sensor 14, the flow volume of the infusion solution caused only by the impact of gravity, i.e., the flow volume that is unaffected by the operation of the infusion solution pump 13.

Next, the CPU 40 causes the infusion solution pump 13 to resume operation, and causes the constricting unit 15 to reduce the flow volume of the infusion solution caused by the impact of gravity applied on the medicinal solution flowing through the tube 23. The constricting unit 15 constricts the tube 23 such that the measured flow volume while the infusion solution pump 13 is driven becomes at least the set flow volume (step S111).

Subsequently, after continuing the drip infusion for a while and the measured flow volume becomes lower than the set flow volume (YES in step S112), it is considered that the medicinal solution bottle 10 has returned to its original position and the flow of the infusion solution is no longer affected by gravity. Therefore, the CPU 40 uses opening/closing control signals for controlling the constricting unit 15 to release the constriction (step S113). Then, the process returns to step S103 and regular operation is continued.

When the measured flow volume does not become lower than the set flow volume (NO in step S112), the process returns to step S103 and regular operation is continued.

If there is no constricting unit 15, when the value of the flow volume sensor 14 is not the set flow volume, or when the value of the flow volume sensor 14 is not an abnormal value but exceeds the range controllable by adjusting the discharge amount of the infusion solution pump 13, there is no other option but to stop operating the infusion solution pump 13. However, by providing the constricting unit 15, it is not only possible to adjust the discharge amount of the infusion solution pump 13, but it is also possible to reduce the flow volume at the end of the flow path. Accordingly, it is possible to increase the extent and freedom in the operation of controlling the flow volume performed by the infusion pump system (infusion apparatus 1).

In the first control operation described above, when it is determined that the flow volume cannot be controlled by the infusion solution pump 13 (step S109), the constricting unit 15 starts operating to control the flow volume. Therefore, there may be a time period during which the flow volume cannot be controlled for the patient.

Therefore, in a second control operation described below, the increasing rate of the flow volume is used as a reference for determining whether the flow volume can be controlled only with the use of the pump.

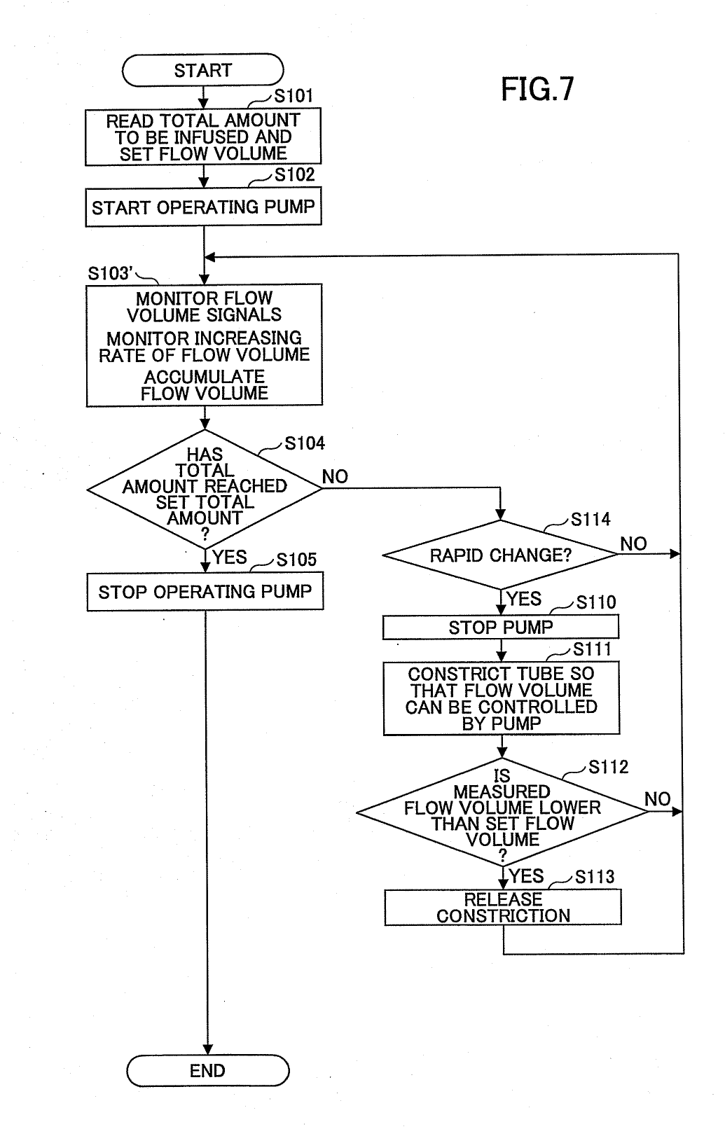

FIG. 7 is a flowchart of the second control operation of the infusion pump system (infusion apparatus 1) according to an embodiment of the present invention.

In the second control operation illustrated in FIG. 7, the timing of taking a measure to control the flow of the infusion solution caused by gravity is different from that of the first control operation.

The CPU 40 monitors flow volume signals (measured flow volume), and accumulates the flow volumes, and also calculates the increasing rate of the flow volume (step S103′). The flow volume usually varies to some extent, but the usual variation amount is within a predetermined rage.

In the present embodiment, the increasing rate of the flow volume is calculated, and when a rapid variation is observed, the infusion solution pump 13 is stopped, and the same measure as that taken in the first control operation is taken, with respect to the flow of the infusion solution caused by gravity. Specifically, when the measured flow volume rapidly increases (the variation of the measured flow volume exceeds a threshold), it is considered that the infusion solution is flowing due to the impact of gravity. By starting the control operation from the time point when the variation of the measured flow volume exceeds the threshold, it is possible to reduce the time taken to control the flow volume.

Incidentally, as described above, when the system controller SC cannot obtain any signals from the flow volume sensor 14, or when the measured flow volume indicates an abnormally high value that is usually inconceivable, it is highly likely that an external failure has occurred in an element of the infusion pump system (infusion apparatus 1).

In such a case, the CPU 40 (interruption control unit 61) interrupts the control operations of the first and second control operations. In this case, the infusion solution is stopped even if a program is being executed by any of the elements. The stopping process includes stopping the operation of the infusion solution pump 13 to stop the infusion solution in the infusion solution pump 13 itself, and instructing the constricting unit 15 to block the flow path.

Furthermore, the CPU 40 causes the announce unit 44 to blink or to produce a sound, or uses the wireless communications unit 43 to send a report to a terminal device (external device) that is held by a nurse.

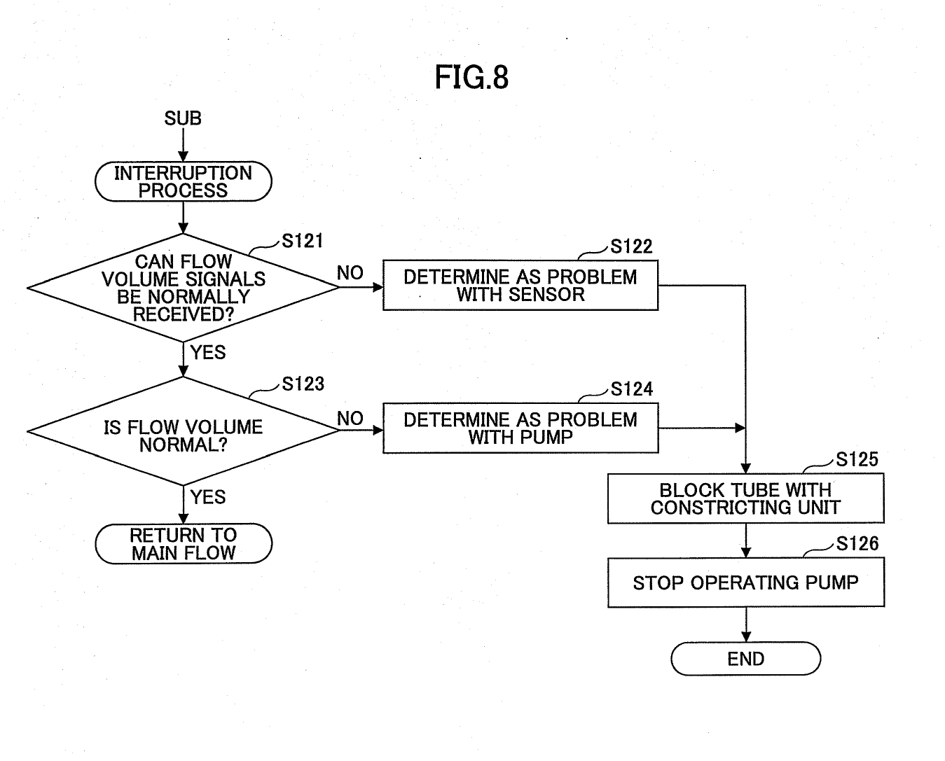

FIG. 8 is a flow chart of a process of performing interruption control when an abnormality occurs.

When the system controller SC can normally receive flow volume signals from the flow volume sensor 14 (YES in step S121), the system controller SC determines that there is no problem with the flow volume sensor 14. Furthermore, when the flow volume is within a normal range (YES in step S123), the system controller SC determines that there is no problem with the infusion solution pump 13. In these cases, the process returns to the main routine as described with reference to FIGS. 6 and 7.

When the system controller SC cannot normally receive flow volume signals from the flow volume sensor 14 (for example, flow volume signals cannot be received at all or the signals indicate a lower voltage than a predetermined voltage) (NO in step S121), the system controller SC determines that there is a problem with the flow volume sensor 14 (step S122). Even when the system controller SC can normally receive the flow volume signals, when the observed flow volume is less than or equal to a threshold (e.g., the flow volume is excessively low or the flow volume is zero, or the flow volume is so high that it cannot be adjusted by controlling the infusion solution pump 13 or by using the constricting unit 15) (NO in step S123), the system controller SC determines that there is a problem with the infusion solution pump 13 (step S124).

Furthermore, there may be an impact on the elements such that the tube is obstructed, the needle falls out, or extravascular administration is performed, or there may be external factors such as the temperature.

In these cases, the interruption control unit 61 causes the constricting unit 15 to block the tube 23 (step S125) and cause the infusion solution pump 13 to stop operating (step S126).

As described above, the constricting unit 15 is provided at the discharging side of a component closest to the part of the infusion apparatus 1 connected to the patient. Therefore, even if the component breaks, the tube (the tube 23 in FIG. 1) directly connected to the blood vessel of the patient can be blocked, so that the infusion solution is prevented from being exposed to external air.

When the system controller SC causes the system controller SC to block the tube 23 in step S125, the system controller SC uses a speaker (not shown) to produce a sound or uses the wireless communications unit 43 to send a report to the nurse.

Furthermore, when the constricting unit 15 blocks the tube 23, the constricting unit 15 sends a report to the system controller SC. Accordingly, the abnormality in the infusion pump system (infusion apparatus 1) is surely reported to the nurse and the patient.

Furthermore, in an embodiment of the present invention, the constricting unit 15 detects whether the system controller SC is operating, and when the constricting unit 15 detects that the system controller SC is not operating, the constricting unit 15 autonomously operates and blocks the flow path.

When the system controller SC is operating, the system controller SC inputs, to the constricting unit 15, signals indicating that the system controller SC is operating (hereinafter, “operation signals”). While such signals are being input, the constricting unit 15 does not perform any operations of blocking the flow path.

When the system controller SC stops operating due to some problem (in the worst case because the power source is cut off), it is assumed that all signals output from the system controller SC including the operation signals become LOW. In this case, the constricting unit 15 blocks the flow path in response to detecting LOW signals.

Furthermore, when the system controller SC stops operating due to an emergency, the constricting unit 15 cannot expect to receive power from the system controller SC. Therefore, the constricting unit 15 is preferably equipped with batteries having sufficient capacity for performing at least the operation of blocking the flow path.

Under normal conditions, the constricting unit 15 receives normal signals from the system controller SC, and thus maintains a constant charged state. Under emergencies, the constricting unit 15 preferably performs the blocking operation with the use of the charged power. Accordingly, the constricting unit 15 can block the flow path even when the system controller SC is shut down.

Furthermore, in order to reliably operate the constricting unit 15, the blocked state may be the regular state, and the flow path may be opened when an instruction is received from the system controller SC as the infusion pump system 1 starts operating.

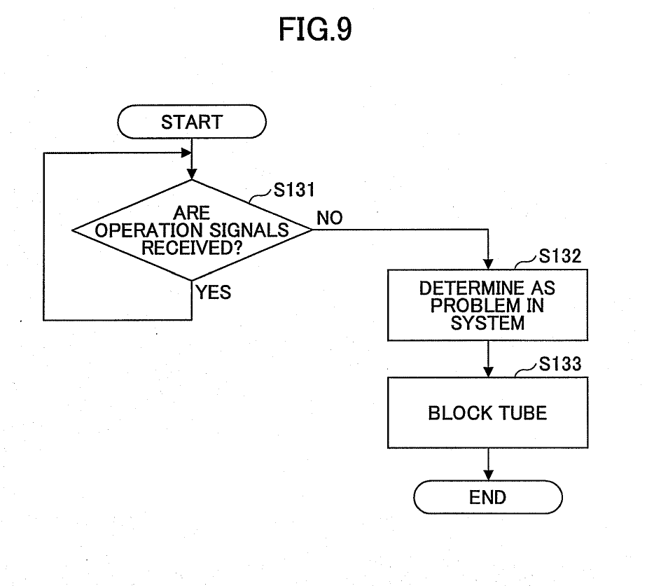

FIG. 9 is a flow chart of an operation performed by the constricting unit 15 when the system controller SC is not operating.

When the constricting unit 15 cannot receive any operation signals (No in step S131), the constricting unit 15 determines that a problem has occurred in the system controller SC (step S132), and blocks the tube 23 (step S133).

Furthermore, when starting the drip infusion operation, before operating the infusion solution pump 13, the system controller SC performs a diagnosis whether the constricting unit 15 can block and open the tube 23. When the constricting unit 15 does not output a signal indicating that the constricting unit 15 has blocked the tube 23, the system controller SC determines that there is an abnormality. Accordingly, the system controller SC causes the announce unit 44 to blink or to produce a sound, or uses the wireless communications unit 43 to send a report to a terminal device (external device) that is held by a nurse. Hence, it is possible to prevent an abnormal drip infusion apparatus from being used beforehand, so that drip infusion can be performed more safely.

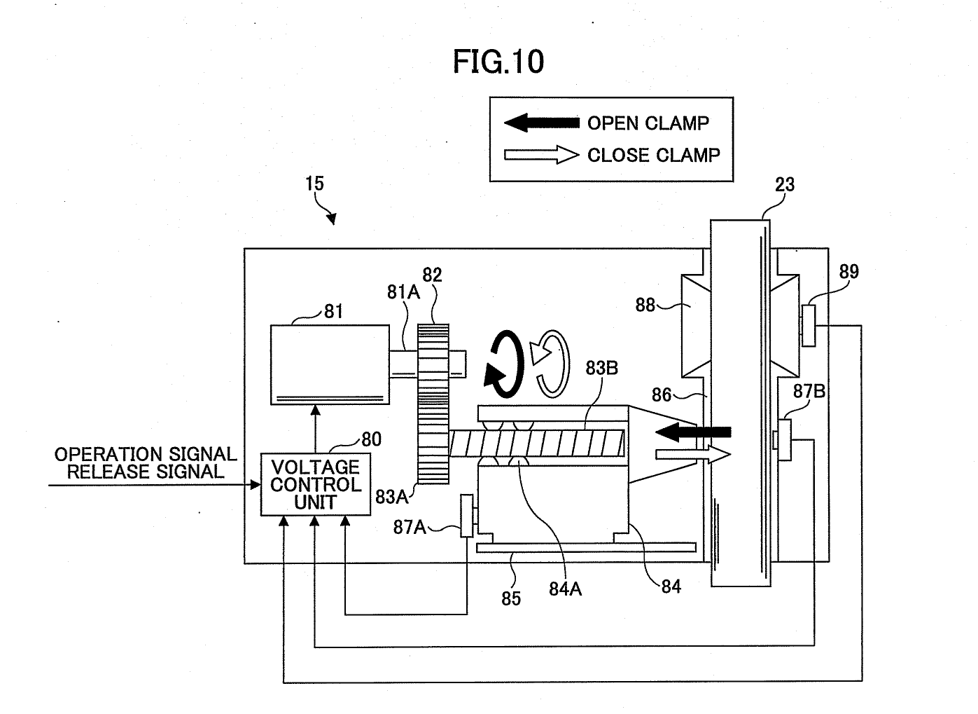

FIG. 10 illustrates a specific example of a flow path resistance changing means for constricting the tube 23.

The constricting unit 15 acting as a flow path resistance changing means includes a stepping motor 81; a first rotational gear 82 attached to a rotational shaft 81A of the stepping motor 81; a second rotational gear 83A that rotates by receiving the rotational force of the first rotational gear 82; a male screw 83B attached to the rotational center shaft of the second rotational gear 83A so as to extend in the opposite direction to the stepping motor 81; and a voltage control unit 80 such as an IC chip for changing the rotation direction of the stepping motor 81 by switching the voltage of the stepping motor 81.

The voltage control unit 80 receives operation signals and release signals from the system controller SC. The constricting unit 15 includes a guide rail 85 having a groove-shaped cross-sectional view. A clamper 84 is attached in such a manner as to freely move along the groove of the guide rail 85.

The clamper 84 has a female screw 84A that is screwed together with the male screw 83B. Accordingly, by driving the stepping motor 81 to rotate the male screw 83B, the male screw 83B changes its position along the axial direction with respect to the female screw 84A of the clamper 84 according to the rotation direction of the male screw 83B. Consequently, the clamper 84 slides by being guided by the guide rail 85.

The constricting unit 15 has a first pressing force sensor 87A for detecting the pressing force from the clamper 84. When the clamper 84 slides toward the stepping motor 81 and presses the first pressing force sensor 87A, the first pressing force sensor 87A detects that it has been pressed by the clamper 84.

The signals output from the first pressing force sensor 87A are transmitted to the voltage control unit 80. As the voltage control unit 80 stops the voltage pulse supplied to the stepping motor 81, the stepping motor 81 stops operating.

Furthermore, the constricting unit 15 includes an insertion hole for inserting the tube 23. On the opposite side of the clamper 84 with respect to the insertion hole, a second pressing force sensor 87B is provided. When the clamper 84 slides and presses the tube 23 inserted in the insertion hole, the diameter of the tube 23 deforms and the tube 23 on the downstream side is constricted, and the tube 23 deforms toward the second pressing force sensor 87B. Accordingly, the second pressing force sensor 87B detects that it has been pressed by the tube 23.

Furthermore, a detector 88 is provided on the outer periphery of the insertion hole in the constricting unit 15. The inner radius of the detector 88 is somewhat smaller than the outer radius of the tube 23. Accordingly, when the tube 23 is inserted into the insertion hole, the tube 23 somewhat pushes out the detector 88, and the tube 23 is gripped by the force of the detector 88 that tries to return to its original shape. Furthermore, on the outer periphery of the detector 88, there is provided a third pressing force sensor 89. The detector 88 that has been somewhat pushed out by the inserted tube 23 detects that the third pressing force sensor 89 has been pressed.

The signals output from the third pressing force sensor 89 are transmitted to the voltage control unit 80. In this case, even if the voltage control unit 80 cannot receive the operation signals from the system controller SC, the voltage control unit 80 starts supplying voltage pulses to the stepping motor 81, and the clamper 84 starts sliding to press the tube 23. Furthermore, when signals are not transmitted from the third pressing force sensor 89 to the voltage control unit 80, it means that the tube 23 is not inserted in the constricting unit 15.

In this case, even if the voltage control unit 80 cannot receive the operation signals from the system controller SC, the voltage control unit 80 does not supply voltage pulses to the stepping motor 81. When the voltage control unit 80 receives release signals described above, the voltage control unit 80 supplies voltage pulses to the stepping motor 81 to slide the clamper 84 in a direction in which the constriction to the tube 23 is released.

The present invention is not limited to the specific embodiments described herein, and variations and modifications may be made without departing from the scope of the present invention.

The present application is based on Japanese Priority Patent Application No. 2010-252706, filed on Nov. 11, 2010, the entire contents of which are hereby incorporated herein by reference.

1. A pump module comprising:
a case; and
a pump that sends a solution in accordance with a change in a volume of a space where the solution flows in and out, the space being included in the pump and the pump being located inside the case,
wherein the space includes
a first opening part through which the solution flows into the space, and
a second opening part through which the solution flows out from the space,
the case includes
a third opening part through which the solution flows into the case, and
a fourth opening part through which the solution flows out from the case,
the space and the case are configured such that the solution flows from the first opening part toward the second opening part,
the fourth opening part is located at a different position from the third opening part,
an angle between a direction in which the solution flows through the third opening part and a direction in which the solution flows through the second opening part is greater than or equal to zero degrees and less than 90 degrees,
the third opening part and the first opening part are connected by a first flow path through which the solution flows into the case and the space,
the second opening part and the fourth opening part are connected by a second flow path through which the solution flows out from the space and the case, and
the second opening part of the space is located closer to the third opening part of the case than is the first opening part of the space.

2. The pump module according to claim 1, wherein the fourth opening part is provided in a wall of the case that is furthest away from the third opening part, among walls of the case.

3-4. (canceled)

5. An infusion pump system comprising:
an infusion solution container filled with an infusion solution;
a needle used for injecting the infusion solution in a biological body; and
a pump module that sends the infusion solution toward the needle, the pump module including
a case, and
a pump that sends the infusion solution in accordance with a change in a volume of a space where the infusion solution flows in and out, the space being included in the pump and the pump being located inside the case,
wherein the space includes
a first opening part through which the infusion solution flows into the space, and
a second opening part through which the infusion solution flows out from the space,
the case includes
a third opening part through which the infusion solution flows into the case, and
a fourth opening part through which the infusion solution flows out from the case,
the space and the case are configured such that the solution flows from the first opening part toward the second opening part,
the fourth opening part is located at a different position from the third opening part,
an angle between a direction in which the infusion solution flows through the third opening part and a direction in which the infusion solution flows through the second opening part is greater than or equal to zero degrees and less than 90 degrees,
the third opening part and the first opening part are connected by a first flow path through which the infusion solution flows into the case and the space,
the second opening part and the fourth opening part are connected by a second flow path through which the infusion solution flows out from the space and the case, and
the second opening part of the space is located closer to the third opening part of the case than is the first opening part of the space.
